(19) United States
(12) Patent Application Publication
(10) Pub. No.: US 2016/0058316 A1
(43) Pub. Date: Mar. 3, 2016

(54) STRONG, CONDUCTIVE CARBON NANOTUBE ELECTRODES

(71) Applicant: WILLIAM MARSH RICE UNIVERSITY, Houston, TX (US)

(72) Inventors: Flavia Vitale, Houston, TX (US); Caleb Tilo Kemere, Houston, TX (US); Matteo Pasquali, Houston, TX (US)

(21) Appl. No.: 14/783,908

(22) PCT Filed: Apr. 14, 2014
(86) PCT No.: PCT/US14/34019

§ 371 Date: Oct. 12, 2015

Related U.S. Application Data
(60) Provisional application No. 61/811,437, filed on Apr. 12, 2013.

(51) Int. Cl. A61B 5/04; A61N 1/05; C01B 31/02; A61B 5/145

(52) U.S. Cl. CPC A61B 5/04001; A61B 5/14546; A61B 5/14503; A61N 1/0534; A61N 1/0551; C01B 31/0253; C01B 2202/02; C01B 2202/08; C01B 2202/22

(57) ABSTRACT

In some embodiments, the present disclosure pertains to a device comprising at least one implantable microelectrode. In some embodiments, the implantable microelectrode comprises at least one fiber of aligned carbon nanotubes partially coated with a layer of biocompatible insulating material. In some embodiment of the present disclosure, at least one end of the fiber of aligned carbon nanotubes is uncoated. In some embodiments, the uncoated end of the fiber is electrically active. In some embodiments, the device further comprises a removable inserting device attached to the implantable microelectrode. In some embodiments, the present disclosure pertains to a method of implanting an implantable microelectrode into a subject. In some embodiments, the present disclosure relates to a method of fabricating an implantable microelectrode.

### CROSS REFERENCE TO RELATED APPLICATIONS

This application claims priority to U.S. Provisional Patent Application No. 61/811,437, filed on Apr. 12, 2013. The entirety of the aforementioned application is incorporated herein by reference.

### BACKGROUND

Electrical stimulation of neural activity generally utilizes electrodes that deliver the required amount of charge to initiate a functional response in a neural structure. Existing electrodes have poor electrochemical properties that limit their use for safely delivering the stimulating charge and reliably recording neural activity at a single unit level. Consequently, a need exists for the development of microelectrodes capable of safely modulating, stimulating and recording the activity of neural ensembles.

### SUMMARY

In some embodiments, the present disclosure pertains to a device comprising at least one implantable microelectrode. In some embodiments, the implantable microelectrode comprises at least one fiber of aligned carbon nanotubes partially coated with a layer of biocompatible insulating material. In some embodiments of the present disclosure, at least one end of the fiber of aligned carbon nanotubes is uncoated. In some embodiments, the uncoated end of the fiber is electrically active. In some embodiments of the present disclosure, the at least one fiber of aligned carbon nanotubes is formed by wet-spinning or direct spinning. In some embodiments, the aligned carbon nanotubes are single-walled carbon nanotubes. In some embodiments of the present disclosure, the biocompatible insulating material comprises polystyrene-polybutadiene. In some embodiments, the device further comprises a removable inserting device attached to the implantable microelectrode. In some embodiments, the removable inserting device is a polyimide wire. In some embodiments, the removable inserting device is attached to the implantable microelectrode by a dissolvable coating. In some embodiments, the dissolvable coating is a polyethylene glycol (PEG) coating. In some embodiments, the implantable microelectrode is a stimulating electrode. In some embodiments, the implantable microelectrode is a sensory electrode at a single neuron level.

In some embodiments, the present disclosure pertains to a method of implanting an implantable microelectrode into a subject. In some embodiments, such a method comprises providing at least one implantable microelectrode and implanting the at least one implantable electrode into the subject. In some embodiments, the at least one implantable microelectrode comprises at least one fiber of aligned carbon nanotubes partially coated with a layer of biocompatible copolymer. In some embodiments, at least one end of the fiber is uncoated. In some embodiments, the method comprises implanting the at least one implantable microelectrode into the subject. In some embodiments, the method further comprises a step of attaching the implantable microelectrode to a removable inserting device. In some embodiments, the removable inserting device is a polyimide wire. In some embodiments, the removable inserting device is attached to the implantable microelectrode by a dissolvable coating. In some embodiments, the dissolvable coating is a polyethylene glycol (PEG) coating. In some embodiments, the implantable microelectrode is a stimulating electrode for microscale neural ensembles. In some embodiments, the implantable microelectrode is a sensory electrode at a single neuron level.

In some embodiments, the present disclosure relates to a method of fabricating an implantable microelectrode. In some embodiments, such a method comprises forming a fiber of aligned carbon nanotubes. In some embodiments, such a method further comprises partially coating the formed fiber of aligned carbon nanotubes with a layer of a biocompatible insulating material such that at least one end of the fiber is uncoated. In some embodiments, the uncoated end of the fiber is electrically active. In some embodiments, the method further comprises the step of attaching the implantable microelectrode to a removable inserting device.

### BRIEF DESCRIPTION OF THE FIGURES

FIGS. 1A-1B show In vitro characterization of CNTf microelectrode properties and comparison with other electrode materials: Modulus (FIG. 1A) and phase of the impedance of CNT fiber and PtIr wires (diameter: 18 μm, red dots CNTf, blu squares PtIr) (FIG. 1B). Specific impedance (FIG. 1C); cyclic voltammetry of CNTf and PtIr electrodes used for in vivo deep brain stimulation study, showing the higher charge storage capacity of CNT fibers (red CNT fiber, blue PtIr) (FIG. 1D).

FIGS. 2A-2K depict in-vivo study of CNT fibers as stimulating electrodes. FIG. 2A shows CNT fiber coated with PSS-b-PBD; FIG. 2B shows two channel CNT fiber microelectrodes used for acute histology and deep brain stimulation studies. FIGS. 2C-2F is an illustration of the implant strategy of CNT fibers in 0.6% agar phantom: CNT fiber microelectrode is attached to the stiffener with PEG adhesive. The stiffener allows the insertion of CNT fiber in the target area (FIG. 2C-2D); PEG dissolves within few minutes after implantation, allowing the removal of the electrode, while CNT fiber electrode is left in place (FIG. 2E-2F).

FIGS. 2G-2H depict the histological analysis of the acute damage to the blood brain barrier (BBB) due to electrode insertion: CNT fiber microelectrode at the entry location (FIG. 2G), and at the tip (FIG. 2H); PtIr electrode at the entry location (FIG. 2I) and at the tip (FIG. 2J). FIG. 2K shows the characteristic length scale of bleeding. (Scale bar 100 μm).

FIGS. 3A-3B show in-vivo characterization of CNT fiber microelectrodes for stimulation of deep brain structures (DBS): 6-OHDA dopaminergic lesion was induced on the right hemisphere (FIG. 3A). CNT fiber electrodes were implanted in the entopeduncular nucleus (EP) ipsilateral to the lesion. Commercial PtIr electrodes were implanted in the left EP, and used as control; FIG. 3B shows the results of the metamphetamine rotation test: average normalized rotation rate of a population of 4 Long-Evans rats implanted with CNT fiber electrodes and comparison with PtIr electrodes (error bars: ±SEM) (FIG. 3B). Repeated measures ANOVA showed that there was significant difference between treatment conditions (p<0.05). Pairwise comparison across frequencies was performed with post-hoc least square difference (LSD, p<0.05). Frequencies are significantly different when do not share a letter.

FIGS. 4A-4I show histological analysis of tissue response to chronic implants of CNTf and PtIr electrodes. FIG. 4A-4B show tissue response after in GPi after six weeks of implant with CNT fiber, also used for deep brain stimulation, and a PtIr electrode implanted contralaterally. Tissue was stained for astrocytes, microglia (top row); activated, ‘pro-inflammatory’ and ‘anti-inflammatory’ macrophages (second row); laminin (third row) and neuronal nuclei (bottom row). Scale bar 500 μm. FIG. 4C-4H show fluorescence intensity profiles at increasing lateral distance from electrode tract: astrocytes (FIG. 4C), microglia (FIG. 4D), activated macrophages (FIG. 4E), ‘pro-inflammatory’ macrophages (FIG. 4F), ‘anti-inflammatory’ macrophages (FIG. 4G), and laminin (FIG. 4H). Error bar: S.E.M. neuronal count at increasing lateral distance from electrode tract (FIG. 4I).

FIGS. 5A-5C show electrochemical characterization of CNT fibers. FIG. 5A shows cyclic voltammogramm recorded by sweeping the potential between the voltage limits of −2 to 2 V (vs. Ag/AgCl electrode). The water window is delimited by the water oxidation and reduction voltages, where a steep increase in the resistive current is observed. The water window of CNT fibers ranges from −1.5 to 1.5 V; FIG. 5B shows voltage excursion in response to a biphasic, charge balanced current pulse of amplitude 100 μA, pulse duration 60 μs and frequency 130 Hz (shown in FIG. 5C). The insets in the plot show the instantaneous voltage drop caused by the resistance of PBS solution (Vacc) and the total voltage magnitude (Vtot) used to calculate the charge storage capacity of CNT fibers.

FIGS. 6A-6F show stability of CNT fibers and PEDOT under prolonged overpulsing. FIG. 6A shows electrode impedance at 1 kHz. CNT fibers show an initial decrease of impedance, already following the first hour of immersion in PBS without voltage pulsing. After 1 hour of voltage pulsing, the impedance further decreases to almost 10% of the initial value. After this initial transient, the impedance remains constant throughout the entire duration of the experiment. A consequent 10 fold increase of the charge accumulated at the electrode interface was calculated from the cyclic voltammogramm (FIG. 6B) (Bars show mean±SD). Such improvement of the electrode impedance had been observed in other CNT-based electrodes and could be explained with a combined “polishing” effect: the immersion and pulsing that causes the removal of particles absorbed on the fiber surface, thus effectively increasing the interfacial area between CNT fiber and the electrolyte.

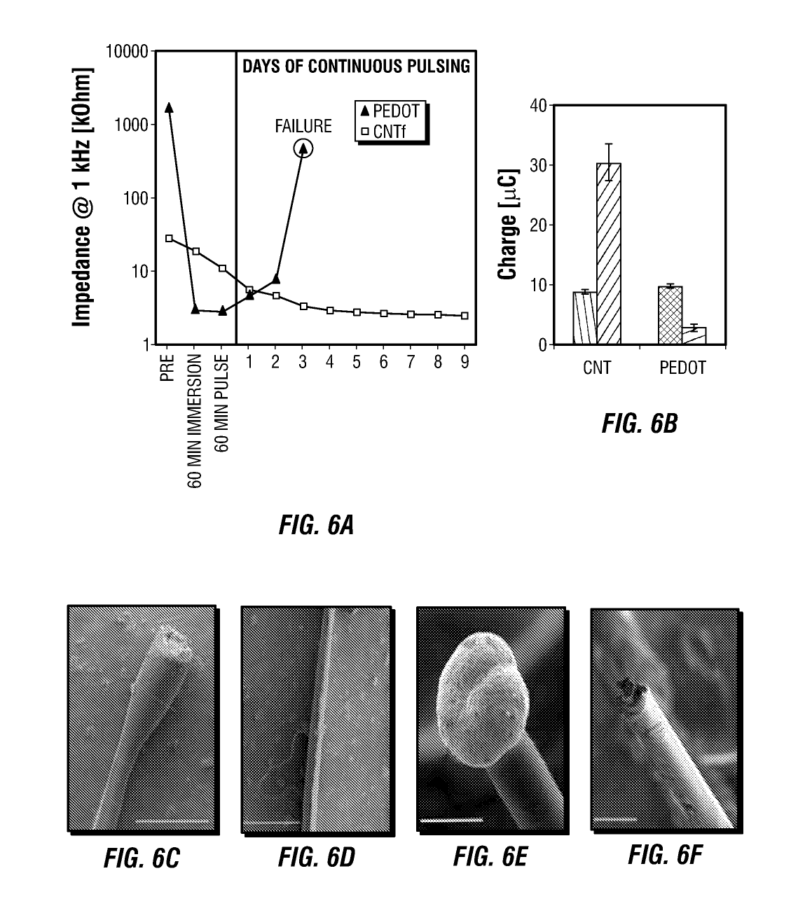

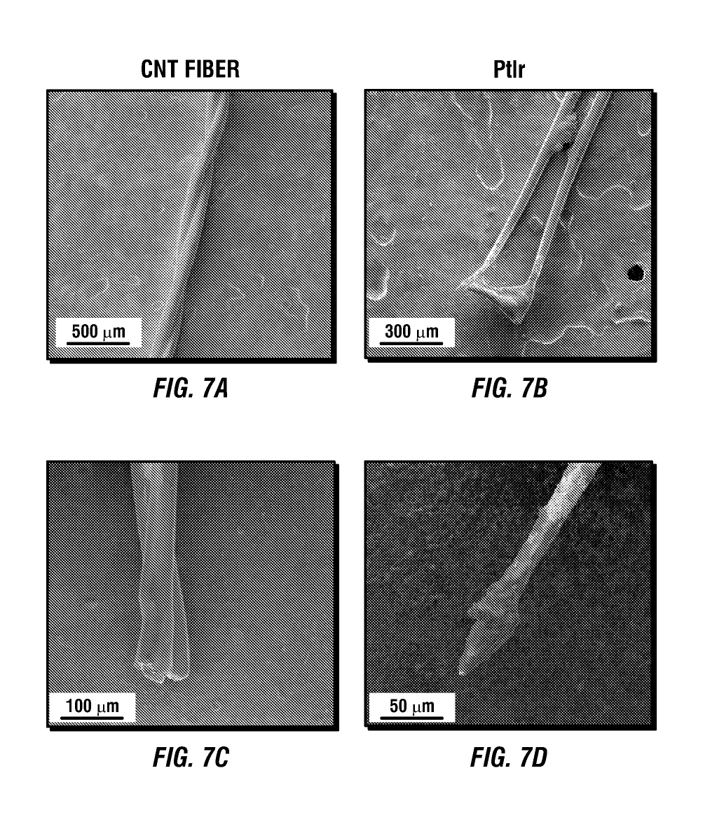

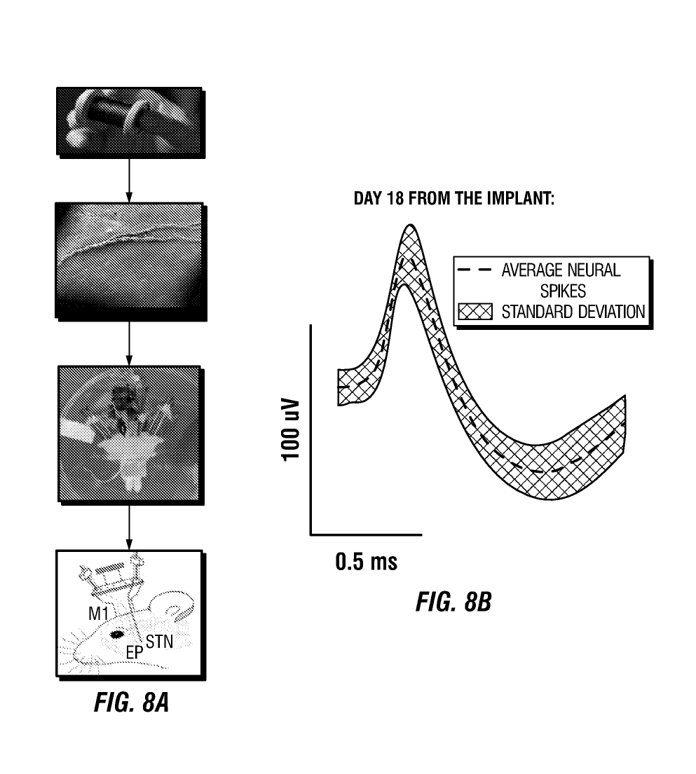

Moreover, SEM imaging of the CNT fiber after 9 days of continuous pulsing shows some fraying at the fiber, which could be partially caused by the pulsing. The continuous pulsing could have further contributed to the increase of the effective surface area, but no evidence of damage of the fiber insulation was observed (FIGS. 6C-6D) (scale bar: 200 μm). The experiment was interrupted after 9 days of continuous stimulation. PEDOT coated electrodes showed increase of impedance after the first day of stimulation. This increase of impedance corresponds to the beginning of pulse-induced degradation of the coating. The complete failure of the coating is indicated by the return of the impedance to the values measured for PtIr wire prior to deposition of PEDOT and was observed at day 4, after 43 millions of cycles, when the experiment was stopped. SEM imaging of the PtIr wire coated with PEDOT-PSS as shown immediately after deposition and before the beginning of the pulsing experiments (FIG. 6D) (scale bar: 20 μm) and after 4 days of continuous pulsing (FIG. 6E). The failure of the PEDOT coating is evident, which was no longer present on the wire tip (scale bar: 50 μm) (FIG. 6F).

FIGS. 7A-7D show SEM microscopy of CNT fibers (FIG. 7A and 7C) and PtIr electrodes (FIG. 7B and 7D), after six weeks of implant in the GPi of two different rats. The formation of cellular aggregates and encapsulation around the tip of PtIr electrodes is evident.

FIGS. 8A-8B show in-vivo recording experiments in the motor cortex of rats implanted with CNT fiber electrodes. FIG. 8A shows the steps of implantation of CNT fiber electrode in the motor cortex of a rat. FIG. 8B shows a recording of the activity of a single neuron in the motor cortex of the rat. The solid curve indicates the mean spike waveform recorded from the CNT fiber channel of the recording tetrode. At each time that a spike occurs, 40 samples of the filtered local field potentials (LFP) signals are saved to produce a spike waveform. These waveforms are noisy and variable, but when averaged r, an approximation of the true neuronal spike waveform is obtained.

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The present disclosure pertains to the use of carbon nanotube (CNT) fibers as materials for recording and stimulating the activity of neural ensembles. In the present disclosure, the applicants have shown that microelectrodes comprising CNT fibers (also referred to as CNT fiber microelectrodes) have geometrical and electrochemical properties suitable for recording and stimulating the activity of neural circuits. The CNT fiber microelectrodes have optimal electrochemical properties as compared to electrodes made of metals and do not need additional plating or metal coating to achieve the optimal electrochemical properties. Further, the CNT fiber microelectrodes of the present disclosure are as effective as metal electrodes in mitigating behavioral symptoms of neurologic disorders but with more than one order of magnitude smaller surface area and with minimal inflammatory response. Further, the CNT fiber microelectrodes of the present disclosure are capable of obtaining stable recording of a single unit activity over an extended period of time in vivo.

Electrical stimulation of neural activity generally utilizes electrodes to deliver the required amount of charge to initiate a functional response in the neural structures. A desirable characteristic for a stimulating electrode is that it must be able to deliver the necessary amount of charge without exceeding the safety voltage potential limit (namely the “water window”), beyond which an irreversible faradaic hydrolysis reaction occurs in the tissue. Further, a second desirable trait is that the stimulating electrode must be able to remain functional for chronic use without degradation and change in its electrochemical properties, and also be biocompatible.

The charge density of an electrode inversely depends on the effective size of the electrode contact (a.k.a. active site), and thus represents the greatest barrier towards the miniaturization of stimulating electrodes. Many neuro-prosthetic applications require that the same electrode be used for both stimulation and recording, which necessitates the use of small geometric surface area (GSA) electrodes (GSA≈2000 μm²). The poor electrochemical properties of metal components greatly limit the realization of small surface area electrodes that can safely deliver the stimulation charge and reliably record neural activity. As a result, none of the existing electrodes can be used for both stimulation and recording of the activity of neural ensembles. Small electrodes enable high spatial resolution and selectivity of neural responses. Moreover, the minimization of the device footprint and its flexibility may also reduce the inflammatory foreign-body response and the mechanical damage caused by the relative micromotion with brain tissue, thus improving the overall biocompatibility of the implant.

A wide variety of materials for use in neural electrode design have been explored. Platinum (Pt) and platinum-iridium (PtIr) alloys are the most commonly adopted materials for large deep brain structures (DBS) and cochlear implants electrodes, because of the good biocompatibility and resistance to corrosion. However, due to the low charge injection limits (0.05-0.15 mC/cm2), Pt cannot be used for the fabrication of small surface area electrodes. Iridium oxide (IrOx) is a promising alternative to Pt for microelectrodes, since it is biocompatible, stable, and has a low impedance and high charge delivery capacity (2-3 mC/cm²) through the reversible faradaic reaction (Ir3+←→Ir4++e−). However, IrOx electrodes undergo destabilization and delamination when subjected to overpulsing beyond charge density limits, which can cause the release of particles. The aforementioned drawback limits the use of such electrodes in long-term applications.

Current implantable electrodes are made of metal or carbon-based materials. Metal microelectrodes are intrinsically limited in the maximum currents and charge density that can be delivered through capacitive or reversible faradaic mechanisms. Moreover, the impedance of metal microelectrodes is generally high (>1 MOhm) which greatly affects the signal-to-noise ratio and resolution of neural recordings. For deep brain structures (DBS), the use of large electrodes imposed by charge density and safety requirements not only does not allow the precise targeting of stimulation, but also limits the development of novel, closed-loop therapeutic paradigms capable of dynamically adapting stimulation parameters to neural activity.

A widely adopted strategy to increase both the charge injection capacity and the effective surface area of the metal electrodes consists of coating the active site with conductive polymers (CP). Particularly, coating with Poly (3,4-ethylenedioxythiophene) (PEDOT) has attracted much attention, because of high charge injection limits observed among electrode materials. Recently, recordings of single unit activity in the rat motor cortex were acquired from an ultra-small (50 μm²) carbon fiber electrode coated with PEDOT. Despite the promising electrochemical properties, PEDOT coatings share the same limitations of IrOx in terms of degradation, delamination and long-term stability, which critically limits the adoption of PEDOT for chronic stimulation applications. Further, the additional coating layer poses safety issue and increases the risk of harmful toxic effects caused by electrode degradation in the tissue.

Carbon nanotubes (CNT) possess electrochemical, electrical and mechanical properties at the molecular level that, alongside with large surface area and biological stability, make them an ideal material for neural electrode fabrication. CNTs have been used to fabricate microelectrodes for in vitro stimulation of hippocampal neurons, conductive coatings for metal microelectrodes, and for in vitro electrophysiology. Recently, the capability of recording a low frequency signal in the rat motor cortex with a standalone CNT-composite microelectrode has been demonstrated. However, because of the challenges of translating the single molecule properties into microscopic assembly and the difficulties of reliably fabricating CNT electrodes, the potential of CNT for neural electrodes has not been fully explored.

In some embodiments, the present disclosure relates to low impedance, high capacitance microelectrodes comprising carbon nanotube (CNT) fibers. In some embodiments, the CNT fiber microelectrodes disclosed herein have a 100 times lower electrochemical interface impedance than standard metal electrodes and more than two times lower than metal electrodes coated with gold. Moreover, the CNT fibers of the present disclosure can be made thinner than metal wires, which improve the precision of sensing and stimulation. In some embodiments, the diameters of individual CNT fibers disclosed herein may range from about 8 μm to about 100 μm. These fibers may reach strengths of 1 GPa, DC electrical conductivities of 2.9 MS/m, and thermal conductivities of 620 W/mK. Because of this unique combination of electrical conductivity, mechanical strength and cellular-scale cross sectional dimension, the CNT fibers of the present disclosure are optimal materials for functional chronic implantable electrodes for single neuron activity recording and microstimulation, both in peripheral and central nervous systems.

The CNT fibers of the present disclosure may be coated with an insulating material (e.g., a polymer) and processed to fabricate single and multifilament microelectrodes with exceptionally low electrochemical interface (˜10 kOhm) and high charge storage capacity (˜300 mC/cm²). The CNT fibers may also be processed to produce electrodes for in-vivo measurements of concentration of neurotransmitter molecules (i.e. voltammetry). Hence, embodiments of the present disclosure pertain to CNT fibers that possess a unique combination of electrical conductivity, mechanical strength, flexibility and a microscale size for the fabrication of implantable microelectrodes.

In some embodiments, the present disclosure pertains to a device comprising at least one implantable microelectrode. In some embodiments, the at least one implantable microelectrode comprises at least one fiber of aligned carbon nanotubes partially coated with a layer of biocompatible insulating material. In some embodiments, at least one end of the fiber is uncoated. In some embodiments the uncoated end of the fiber is electrically active.

In some embodiments, the device further comprises a removable inserting device attached to the at least one implantable microelectrode. In some embodiments, the at least one implantable microelectrode is a neural stimulating electrode. In some embodiments, the at least one implantable microelectrode is a sensory electrode at a single neuron level. In some embodiments, the at least one implantable electrode is a neural stimulating electrode and a sensory electrode at a single neuron level.

In some embodiments, the present disclosure pertains to a method of implanting an implantable microelectrode into a subject. In some embodiments, such a method comprises providing at least one implantable microelectrode and implanting the at least one implantable electrode into the subject. In some embodiments the at least one implantable microelectrode comprises at least one fiber of aligned carbon nanotubes partially coated with a layer of a biocompatible insulating material. In some embodiments, at least one end of the fiber is uncoated. In some embodiments the uncoated end of the fiber is electrically active.

In some embodiments, the method further comprises a step of attaching the at least one implantable microelectrode to a removable inserting device. In some embodiments, the at least one implantable microelectrode is a neural stimulating electrode. In some embodiments, the at least one implantable microelectrode is a sensory electrode at a single neuron level. In some embodiments, the at least one implantable electrode is a neural stimulating electrode and a sensory electrode at a single neuron level.

In some embodiments, the at least one implantable microelectrode is implanted into a subject by injection. In some embodiments, the at least one implantable microelectrode is implanted into a subject by insertion. In some embodiments, the at least one implantable microelectrode is implanted into the central nervous system of the subject. In some embodiments, the at least one implantable microelectrode is implanted into the peripheral nervous system of the subject. In some embodiments, the at least one implantable microelectrode is implanted into the deep brain structures (DBS) of the subject.

In some embodiments the method is utilized to measure in vivo levels of brain chemicals. In some embodiments the method is utilized to measure in vivo levels of brain chemicals neurotransmitters.

In some embodiments, the present disclosure pertains to a method of fabricating an implantable microelectrode. In some embodiments, such a method comprises, forming a fiber of aligned carbon nanotubes and partially coating the formed fiber of aligned carbon nanotubes with a layer of a biocompatible insulating material. In some embodiments, at least one end of the fiber remains uncoated. In some embodiments, partially coating the fiber includes steps of completely coating the fiber and then removing parts of the coating to expose one end. In some embodiments, partially coating the fiber includes completely coating the fiber and then modifying the fiber to expose one end. In some embodiments, the method further comprises a step of attaching the implantable microelectrode to a removable inserting device.

Various biocompatible insulating materials may be compatible with the device and methods of the present disclosure. In some embodiments, the biocompatible insulating material for coating the at least one fiber of aligned carbon nanotubes may be a polymer or a block copolymer. Examples of suitable polymers that can be utilized as biocompatible insulating materials include, without limitation, poly (p-xylylene), polyimide, polyvinyl alcohol, polytetrafluoroethylene, and combinations thereof. Various block copolymers may be compatible for coating the at least one fiber of aligned carbon nanotubes. Examples of suitable block copolymers that can be utilized as biocompatible insulating materials include, without limitation, polystyrene:polybutadiene (PS-b-PBD), polystyrene:polyisobutylene, and combinations thereof. In some embodiments, the block copolymers are polystyrene:polybutadiene (PS-b-PBD).

In some embodiments of the present disclosure, the at least one implantable microelectrode has specific interface impedance ranging from about 5 Mohm μm² to about 50 Mohm μm². In some embodiments of the present disclosure, the at least one implantable microelectrode has an average impedance of about 10² kOhm at 1 kHz. In some embodiments, the at least one implantable microelectrode is a high capacitance electrode. In some embodiments, the at least one implantable microelectrode has a charge storage capacity of about 310 mC/cm² to about 430mC/cm². In some embodiments, the at least one implantable microelectrode has a diameter ranging from about 8 μM to about 100 μM.

Various removable inserting devices may be compatible with the device and methods of the present disclosure. Examples of inserting devices include but are not limited to wires comprising biocompatible materials and custom designed devices fabricated with biocompatible polymers and metals. In some embodiments the removable inserting device is a polymer-based wire. In some embodiments, the removable inserting device is a polyimide wire.

Various methods may be compatible for attaching the removable inserting device to the at least one implantable microelectrode. In some embodiments, the removable inserting device may be attached to the at least one implantable microelectrode by a dissolvable coating. Examples of dissolvable coatings include but are not limited to polyethylene glycol (PEG), chitosan solution, sucrose solution, and iced water. In some embodiments the dissolvable coating is a polyethylene glycol (PEG) coating. In some embodiments, the implantable microelectrode is attached to the inserting device by a process of dip-coating.

Various methods may be compatible for forming the at least one fiber of aligned carbon nanotubes. In some embodiments, the methods include liquid- and solid-state spinning techniques. Solid-state spinning is usually performed with natural materials, where discrete fibers are spun into a material such as a yarn. In contrast, most synthetic fibers, such as those produced from polymers, are formed from a concentrated, viscous fluid. The viscous fluid may be a melt or solution of the fiber material, which is extruded through flow processing and converted into a fiber through cooling or solvent removal. These two methods have been adapted for spinning of carbon nanotubes into fibers, taking into consideration the inherent properties of carbon nanotubes. In particular, liquid-state spinning of carbon nanotubes has been hampered by carbon nanotubes' high melting points and lack of solubility in normal organic solvents.

In some embodiments, the fibers of aligned carbon nanotubes may be formed by an extruding step. In some embodiments, the extruding step comprises a process selected from a group consisting of wet-jet wet spinning, dry-jet wet spinning and coagulant co-flow. Each of these extrusion processes is considered in more detail below.

In some embodiments, fibers of aligned carbon nanotubes may be spun using a wet-jet wet spinning process similar to that previously described in commonly owned U.S. patent application Ser. No. 10/189,129. The wet-jet wet spinning processes and methods provide enhanced alignment capabilities by utilizing a liquid crystalline solution which can be tensioned after extrusion. In the wet-jet wet spinning process, the extrudate is directly immersed in a coagulant from an extrusion orifice. In various embodiments, the extruding step occurs into at least one coagulant without exposure to atmosphere. Effective coagulants include, but are not limited to, chloroform, dichloromethane, tetrachloroethane and ether. Other methods of processing the carbon nanotube solution in chlorosulfonic acid may be utilized as well.

In various embodiments, the extruding step takes place in an air gap. Instead of direct extrusion into a coagulant as in wet-jet wet spinning, the extrudate may pass through an air gap prior to entering the coagulant. Such a process is referred to as dry jet wet spinning. Processing carbon nanotube articles using a dry-jet wet spinning process can prove advantageous over wet-jet wet spinning. For example, dry-jet wet spun fibers demonstrate an increased density and greater coalescence when exposed to the air gap, compared to comparable fibers prepared by wet-jet wet spinning. Fibers spun in an air gap tend to experience a greater tensioning force relative to fibers spun in solution, which is advantageous for carbon alignment. In certain embodiments, the mechanical properties of dry-jet wet spun articles may be enhanced 10-fold over wet-jet wet spinning.

In various embodiments, the carbon nanotubes that are utilized in the methods and device of the present disclosure are selected from a group consisting of single-wall carbon nanotubes, double-wall carbon nanotubes, multi-wall carbon nanotubes, shortened single-wall carbon nanotubes, and combinations thereof. In some embodiments, the carbon nanotubes have a length up to about 10 mm. In some embodiments, the carbon nanotubes have a length up to about 5 mm. In some embodiments, the carbon nanotubes have a length up to about 1 mm. In some embodiments, the carbon nanotubes have a length up to about 500 μm. In some embodiments, the carbon nanotubes have a length of up to about 500 nm. In some embodiments, the carbon nanotubes are substantially defect free. The relative incidence of defect sites in the carbon nanotubes may be monitored using the G/D ratio obtained from Raman spectroscopy.

The microelectrodes of the present disclosure may be implanted into various subjects. In various embodiments, subjects include animals and humans.

The aforementioned embodiment will be discussed in more detail below. Various aspects of the methods and systems of the present disclosure will also be discussed with more elaboration below as specific and non-limiting examples.

Applications and Advantages

The microelectrodes of the present disclosure show superior specific electrical conductivity than metals and, because of the improved tensile strength, can be fabricated with small diameter (as low as ≈10 μm) without a significant risk of breaking. Small diameter, in turn, allows for increased flexibility, reduced impact and risk of damage to tissue surrounding the implant, and lower GSA. The microelectrodes of the present disclosure may be subjected to bending, forming kinks in the structure, without causing any change in electrical conductivity. Moreover, the microelectrodes of the present disclosure induce less imaging artifacts in MRI compared to PtIr, which is an important tool for post-operative localization of electrodes and general medical diagnostics, as well as to promote neuronal growth and migration.

As such, the microelectrodes of the present disclosure may be used to fabricate implantable electrodes for high-quality recording and low-voltage selective stimulation of neural ensembles. The low stimulation voltage reduces the risks of harmful reactions in the tissue, stimulation artifacts and eliminates the issue associated with electrode degradation. Flexibility and subcellular size enable significant improvements of electrode biocompatibility and lifetime, with minimization of both short-term (i.e., surgical insertion) and long-term (i.e., electrode physiological motion) mechanical trauma to the surrounding tissue.

Variations can be introduced to manipulate electrode properties by engineering the morphology at the electrode/neuron interface. In addition, coating with biodegradable molecules can be introduced for in vivo voltammetry applications. In some embodiments, the biodegradable coating may temporary increase the axial stiffness of the electrode, facilitating the surgical insertion.

### Additional Embodiments

From the above disclosure, a person of ordinary skill in the art will recognize that the methods and systems of the present disclosure can have numerous additional embodiments. Reference will now be made to more specific embodiments of the present disclosure and experimental results that provide support for such embodiments. However, Applicants note that the disclosure below is for exemplary purposes only and is not intended to limit the scope of the claimed invention in any way.

### EXAMPLES

Additional details about the experimental aspects of the above-described studies are discussed in the subsections below.

### Example 1

### Fabrication of CNT Fiber Microelectrodes

CNT fibers were fabricated with a wet-spinning method previously described. In this work, applicants' used CNT fibers with diameter of 13, 18 and 43

μm. Individual filaments of CNT fibers were coated with a 2.4±1.7 μm layer of a copolymer of polystyrene-polybutadiene (PS-b-PBD, Sigma Aldrich), leaving only the tip exposed as an electrically active site. PSS-b-PBD was selected because of the combination of good dielectric properties with biocompatibility, flexibility and resistance to flexural fatigue.

### Example 2

### Electrochemical Characterization

Electrochemical spectroscopy (EIS), cyclic voltammetry (CV) were performed with a Gamry Reference 600 potentiostat (Gamry Instruments, Warminster, PA, USA) in phosphate buffered saline, pH 7.4 (Gibco) at room temperature. A three-electrode configuration was used, with the potentials reference to an Ag/AgCl electrode, a large surface area carbon wire as counter electrode and the tested sample as working electrode. EIS was performed in the frequency range 1-104 Hz at Vrms of 10 mV. Cyclic voltammograms were recorded by sweeping the PtIr electrode between the voltage limits of −0.6 and 0.8 V and the CNT fiber electrodes between −1 and 1 V at scan rate of 0.1 V/s. Each sample was swept for two cycles and the cathodic charge storage capacity was calculated as the time integral of the cathodic current recorded in the second cycle.

For characterization of the electrochemical water window, cyclic voltammetry was performed between the voltage limits of −2 and 2 V and the water oxidation and reduction potentials were determined as the potentials at which sharp peaks in the anodic and cathodic current were detected. Voltage transient experiments were performed with a stimulator AM Systems (Sequim, Wash.). Biphasic, cathodic first, current pulses of 60 μs duration and equal amplitude per each phase were delivered to the tested sample. Pulse frequency was kept at 130 Hz. Voltage transients were recorded with an oscilloscope and the maximum negative potential excursion (Vₘₐₓ) was calculated by subtracting the initial access voltage due to solution resistance from the total voltage (Vₜₒₜ). The charge injection capacity was calculated by multiplying the current amplitude and pulse at which Vₘₐₓ reaches the water reduction limit and diving by the geometric surface area of the electrode. Values are reported as mean±SD.

### Example 3

### Analysis of Stability

Stability analysis under continuous over potential stimulation was performed by immersing CNT fibers in a cell filled with PBS, pH 7.4 (Gibco) at room temperature. Two electrode configuration was used with CNT fiber as working electrode and large surface area carbon wire used as return and reference electrode. The cell was sealed, in order to keep the solution impedance constant, by avoiding evaporation of the electrolyte. CNT fibers were stimulated with phasic voltage pulses with 60 μs/phase duration, pulse amplitude of 3V and frequency 130 Hz, supplied from a National Instruments 4-Channel, 16 bit, ±10 V analog output module (NI-9263) mounted on a CompactDAQ system (NI cDAQ 9174). National Instruments LabVIEW was used to control the voltage generation. Impedance spectra at Vrms of 10 mV and cyclic voltammogramm between −1 and 1 V at scan rate of 0.1 V/s were recorded with a Gamry Reference 600 potentiostat. The electrodes were tested before the beginning of the stability experiments, after 1 hour of immersion in the cell filled with PBS, after 1 hour of voltage pulsing and then in each of the following days after ˜23 hours of continuous stimulation (c: 10.8 M pulses/day). 4 samples were connected to the voltage generator and tested at the same time.

The same protocol described above was used to test the stability of PEDOT-poly(styrene sulfonate) (PSS) deposited on the electrically active site of a PtIr microwire coated with polyimide along the axial length (d=18 μm, California Fine Wire). The electrolyte for PEDOT-PSS deposition consisted of a solution of 0.2% w/v of the monomer EDOT (Sigma-Aldrich), and 0.2% w/v PSS sodium salt in deionized water (DI). The PtIr microwire was immersed in the monomer solution and served as working electrode. Ag/AgCl electrode was used as reference, a large area carbon wire as return electrode and PEDOT-PSS was deposited with a galvanostatic charge of 90 μC, applied with a Gamry Reference 600 potentiostat. After PEDOT-PSS deposition, they were kept immersed in DI until for 1 hour to remove impurities and excess PEDOT. The electrodes were used within the same day of PEDOT-PSS deposition. The potential limits in the case of PEDOT cyclic voltammetry were set at −0.6 and 0.8 V.

### Example 4

### Histology, Imaging and Quantitative Analysis of the Acute Damage to Brain Vasculature and Blood Brain Barrier (BBB)

1,1′-Dioctadecyl-3,3,3′,3′-tetramethylindocarbocyanine (DiI) was used to paint the blood vessels of rats (n=2) and visualize the microvasculature in the brain. The presence of DiI outside of the microvasculature is an indication of a disruption of the blood brain barrier (BBB) since the dye is impermeable to the BBB. The dye was prepared by mixing the crystalline powder in methanol solvent, at a concentration of 6 mg/ml, and then placing it covered on a rocker overnight at room temperature to dissolve; this preparation is consistent with previous work. The mixture was filtered following dissolution of the powder in methanol. Two electrodes were implanted bilaterally in STN (AP −3.6, ML +/−2.6, DV −8.1). A platinum-iridium electrode was implanted in the left hemisphere and a CNT fiber electrode was implanted on the right hemisphere. Following the implantation, the rats received an intravascular (IV) injection of DiI (1 ml of 6 mg/ml in methanol) at a rate of 0.5 ml/min. Immediately following dye injection, the rats received a fatal IP injection of Euthasol. The rats was then transcardially perfused with 100 ml of pH 7.4 phosphate buffered saline (PBS) followed by 250 ml of 4% paraformaldehyde (PFA) to fix the brain tissue. The brain was removed and stored in the same PFA until it sunk in the container. Sucrose was added to create a 30% sucrose solution in PFA and the brain was maintained in this cryoprotective solution until it reached total absorption. The brain was then frozen in Tissue-Tek and kept at −86 degrees Celsius until it was sliced. Frozen tissue was sliced coronally into 30 μm sections using a cryostat machine (microtome) and stored in PBS. For microscopic examination, brain slices were placed on glass slides, covered with cover slips and imaged with Nikon A 1-Rsi Confocal Microscope. Excitation and emission intensities for DiI are 549 nm and 565 nm respectively.

Images were analyzed with a custom written MATLAB script (Mathworks Inc., USA). The midline of the stab wound created by the electrode implant was manually defined. The script calculates the distance of every pixel from this midline and computes the average fluorescence in 10 μm, expanding from the center of the electrode (x=0) tract up to x=±500 μm. To compare the extent of acute damage of CNT fiber with PtIr electrodes, the characteristic length scale of bleeding k was calculated from fitting the fluorescence intensity profiles at both sides of the electrode tract with the function:

$$I ( x ) = a exp ( - \frac{λ}{x} ) \qquad ( 1 )$$

### Example 5

### Animal Surgery

All rats were induced to be hemi-parkinsonian and were implanted with two stimulating stereotrodes, one made from the carbon nanotube fibers (CNTf) and one made from platinum iridium. The subjects received a unilateral injection of 6-OHDA in the right hemisphere, and were implanted with the stereotrodes in the left and right entopeduncular nucleus (EP), the rat equivalent of the GPi. Prior to surgery, desmethylipramine (DMI, 10-20 mg/kg IP), which is a serotonin-norepinephrine reuptake inhibitor (SNRI), was administered to protect noradrenergic neurons. Under anesthesia (0.5-5% isoflurane in oxygen, buprenorphine 0.01-0.05 mg/kg SQ), 6-OHDA (2 μl of 4 mg/ml in 0.9% saline; Sigma, Zwijndrecht, The Netherlands) was stereotactically injected into the medial forebrain bundle (MFB, coordinates from Bregma: AP −4, ML 1.2, DV −8.1). In the same procedure, a platinum iridium stereotrode (R=10 kOhm; MicroProbes, Maryland, USA) was implanted in the contralateral EP (coordinates from Bregma: AP −2.5, ML −3, DV −7.9) and a CNTf stereotrode was implanted in the EP ipsilateral to the 6-OHDA injection. Craniotomies were sealed with silicone elastomer (World Precision Instruments, Florida, USA), and the electrode connectors were affixed in place with 6-12 stainless steel skull screws and dental methacrylate (i.e. acrylic). The solvent for methacrylate is also a solvent for the insulating polymer on the CNTf, so silicone elastomer was also used to form a protective barrier from the acrylic for the exposed CNTf. The rats were given 2 days of post-operative care and all rats began behavior testing began 3 weeks following the injection of 6-OHDA, which is sufficient time for a lesion to develop.

### Example 6

### Drug-Induced Rotation Tests

Methamphetamine dissolved in saline was administered IP (1.875 mg/ml) under isoflurane anesthesia (5% in oxygen). Rats regained consciousness in 1-2 minutes and rested for an additional 15 minutes. This resting period allowed the methamphetamine to take effect in the rats. Rats were then placed in a cylindrical environment (diameter 30 cm, height 45 cm) made of clear acrylic and allowed to behave spontaneously. Infrared video was captured by a Kinect (Microsoft, Washington, USA) and processed in Matlab to determine the angular movement of the rat over time. Each test consisted of two blocks of eight epochs each. One epoch was allocated for testing the rat in the hemi-parkinsonian state (i.e., stimulation was off) and then seven epochs were allocated for the seven different stimulation frequencies ranging from 85 to 175 Hz. Each stimulation epoch was two minutes in duration and was followed by a control epoch that was 3 minutes in duration. The order of the epochs was randomized within each block. The rotation rates during the prior and post control epochs were averaged and used to normalize the rotation rate of the stimulation epoch.

### Example 7

### Chronic Histology

At 6 weeks post-op, subjects (rats) were anesthetized and administered a fatal I.P. injection of Euthasol (0.5-2 ml; Virbac AH Inc.) and then transcardially perfused with 250 ml of a 10% isotonic sucrose solution followed by 250 ml of 4% paraformaldehyde (PFA). The brain was removed and the electrodes were explanted at this time. The tissue was allowed to fix in PFA for 1-2 days at 4 degrees celsius, to ensure complete absorption. Sucrose was then added to create a 30% sucrose solution in PFA to aid in cryoprotection of the tissue and the brain was maintained in this solution at fridge temperature until it sunk in the solution. The tissue was then frozen in Tissue-Tek OCT and kept at −80 degrees celsius until it was sliced. Frozen tissue was sliced coronally into 30 μm sections using a cryostat machine (microtome) and stored in PBS. Sections were then immunostained by incubating in the appropriate primary antibodies: rabbit anti-glial fibrillary acidic protein (GFAP for astrocytes, mouse anti-ionized calcium binding adaptor molecule 1 (Iba1) for microglia, mouse anti-CD68 for activated macrophages, and goat anti-CCR7 for M1 macrophages and rabbit anti-CD206 conjugated to FITC macrophages M2. Integrity of BBB was detected by immunostaining with rabbit anti-laminin.

All the tissue sections were also stained with 4′,6-diamidino-2-phenylindole (DAPI) to mark all cell nuclei.

### Example 8

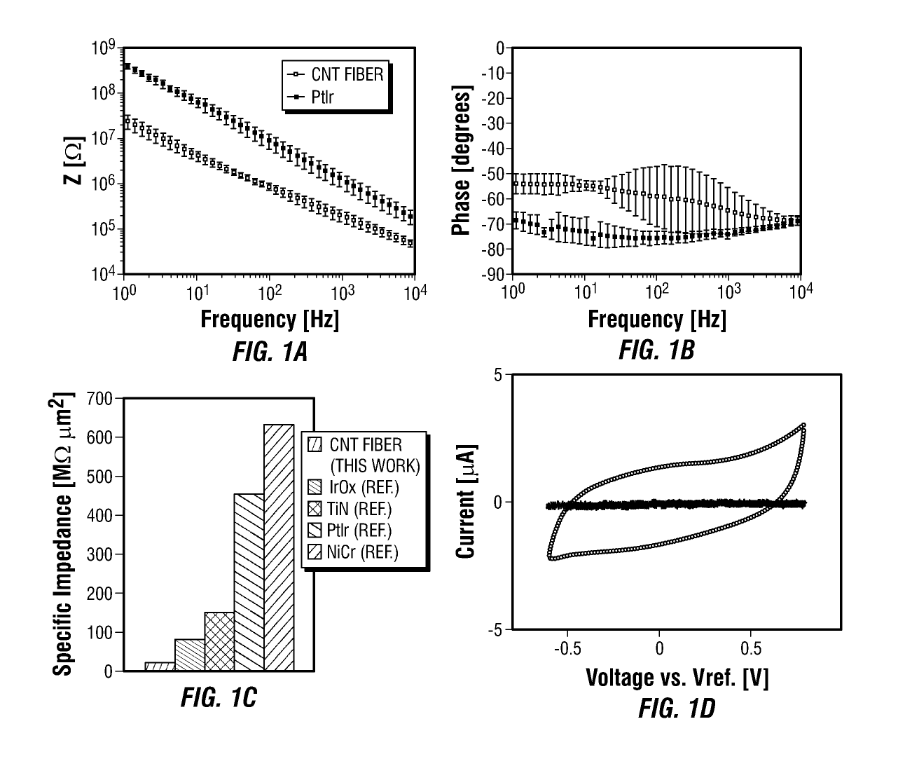

Electrochemical properties of the resultant electrode were characterized through analysis of impedance, charge storage capacity, charge injection limit and the water window. These aspects completely define the space of operation of any implantable electrode and, while specific requirements depend on the application, generally minimization of impedance and maximization of the charge storage and injection properties are considered particularly desirable for achieving noise reduction and stability of recording and safety and efficacy of stimulation. The electrochemical properties of the CNT fiber (CNTf) were measured with electrochemical impedance spectroscopy (EIS) and cyclic voltammetry, in a three-electrode cell filled with phosphate buffered saline PBS (pH 7.4, Gibco) using the CNTf as the working electrode, Ag/AgCl as the reference electrode and a large-surface carbon wire as the counter electrode. The impedance of the CNTf electrode is 15-20 times lower than a PtIr wire of the same size (FIG. 1A) in the range of frequencies tested (1 Hz-10 kHz). This reduction in the interface impedance is confirmed when CNTf are compared with other electrode materials (FIG. 1C), resulting in an average value of the specific interface impedance of 30.6±13.5 MOhm μm². The intrinsic lower specific impedance of CNTf is particularly desirable for single unit recording, because it enables the fabrication of electrodes with an impedance of ˜10²kOhm at 1 kHz (the relevant spiking frequency of neurons) and close to cellular scale size (˜10 μm), without the need of additional conductive plating of the active site. Such improved impedance properties can be attributed to the high effective surface area of CNT fibers, which are composed by bundles of highly aligned CNTs, tightly assembled in the fiber macroscopic structure. The value of the phase lag and the featureless appearance of the cyclic voltammogramm of CNT fibers (FIGS. 1B and 1D) suggest that the nature of the electrochemical interaction is mainly dictated by the capacitive charging and discharging of the CNT fiber-electrolyte double layer. Cathodic charge storage capacity obtained by time integration of cathodic current is 372±56 mC/cm², which is two to three-folds higher than most metal electrodes. Capacitive charge injection is particularly advantageous for neural stimulation applications, since it avoids the risk of tissue damage from irreversible faradaic reactions.

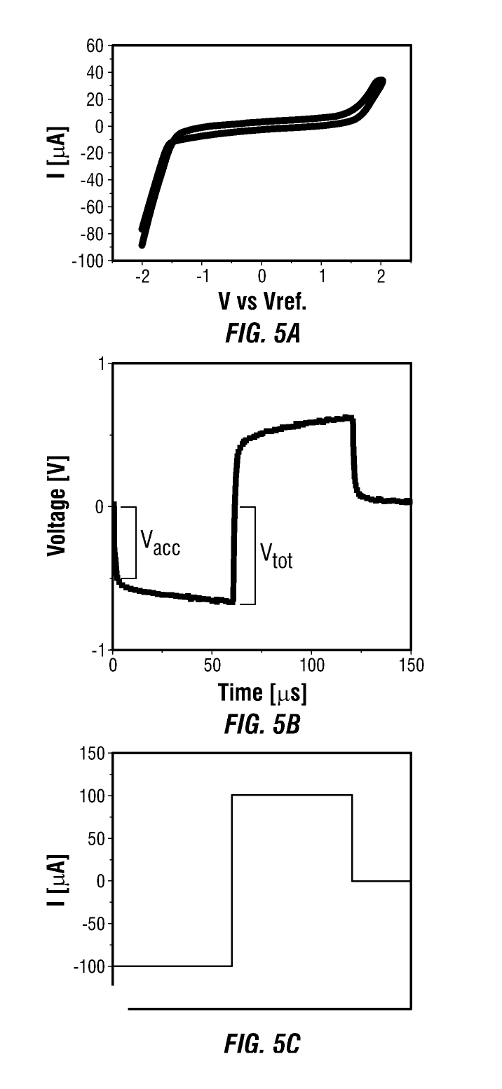

One of the main limitations of stimulating metal electrodes is the low charge density that can be delivered during a stimulating pulse without exceeding the water window electrolysis limits. CNT fibers show a wide water window, with the reduction and oxidation potentials of −1.5 and 1.5 V respectively (FIGS. 5A); the charge injection capacity calculated from the voltage excursion at a conservative maximum negative potential of −1 V is 6.5 mC/cm², which is more than two times higher than most electrode materials. As previously mentioned, the material with the highest charge injection limit is PEDOT, but the adoption of this material for use with stimulating electrodes is limited by stability issues. CNT fibers do not suffer from the same limitation and show not only stability but improvement of impedance properties, even when subjected to 97 M cycles of pulsing beyond the water window limits (9 days), whereas PEDOT shows evidence of coating failure after 43 M of cycles (FIGS. 6A-6F). The wide water window, the higher charge injection capacity and the stability make the CNT fiber a candidate material for the fabrication of recording and stimulating microelectrodes, capable of delivering a high amount of charge without the risk of inducing harmful reactions in the tissue.

### Example 9

Biocompatibility is a factor of primary importance when a material is considered for neural implants. The term biocompatibility refers to the ability of an implant to retain functionality over an extended duration in the host organism, without inducing any adverse or toxic reaction nor degradation of the materials. The response of the brain tissue to the presence of a foreign material can be divided into two phases: the early, acute reaction (duration ˜1-2 weeks) and the chronic inflammatory response (2 weeks to 6 months). In the case of neural microelectrodes, the acute reaction is caused by the trauma from surgical insertion of the electrode and is strongly dependent on the insertion strategy as well as implant size. The stab wound created during surgical insertion may induce disruption of blood vessels and the blood brain barrier (BBB), causing the extravasation of erythrocytes, activation of the coagulation cascade, edema, and accumulation of activated microphages, microglia and astrocytes around the injured area. This initial response serves to protect against inflammation and initiates the wound healing response. However, an excessive extension of the acute lesion can result into a worsening of the chronic inflammation. Thus, the use of flexible microelectrodes can allow for the minimization of both the acute damage and the chronic inflammatory response. While flexibility may lead to enhanced biocompatible features, it can pose issues in terms of precision of electrode localization, mainly when the electrode has to be implanted into deep brain structures (DBS) that are targets of deep brain stimulation (penetration depth ˜8 mm in the adult rat brain).

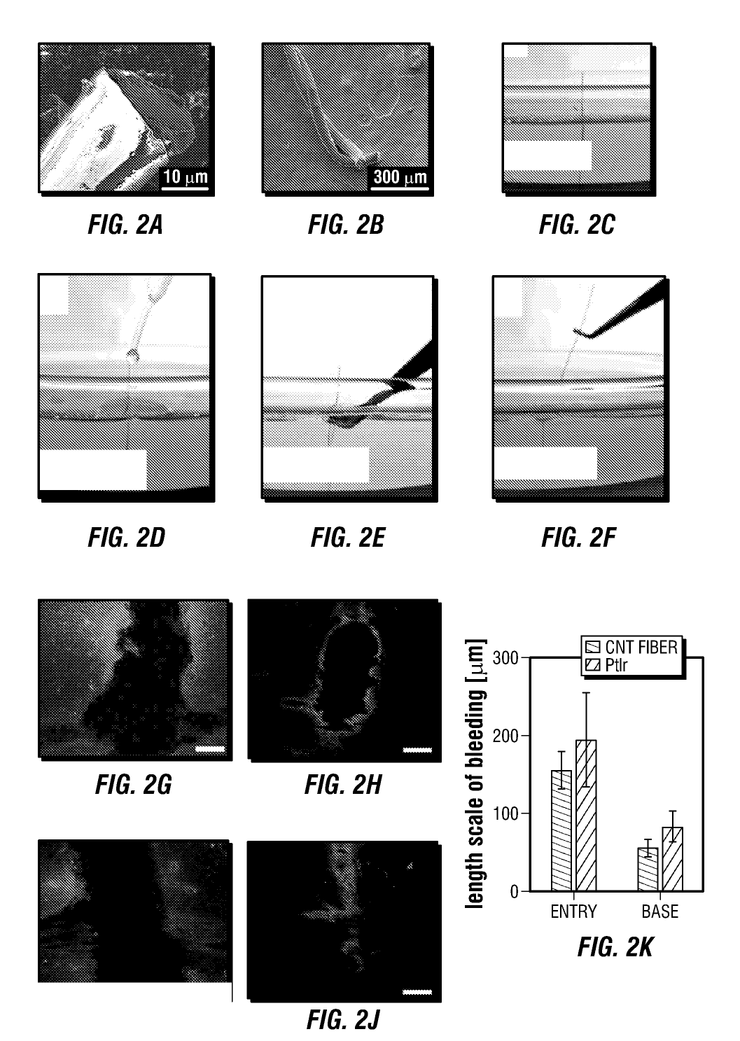

Ideally an electrode should be temporarily stiff to allow for the successful insertion in the target brain area, and flexible in the long term to minimize chronic inflammation. Applicants have developed an ad-hoc surgical insertion procedure which utilized a temporary shuttle to achieve stiffness peri-implantation. Two channel stimulating electrodes (stereotrodes) were fabricated by twisting two PSS-b-PBD coated CNT fibers (FIGS. 2A-2B) with a diameter of 43±4.6 μm and average impedance of 11.2±7.6 kOhm. CNT fiber electrodes were attached to a polyimide (PI) shuttle (diameter 100 μm) via a process of dip-coating in a 5% aqueous solution of biocompatible, water soluble polyethylene glycol (PEG) and air drying; the stereotrodes were sterilized in an ethylene oxide (EO) gas sterilizer and stored until the implantation procedure. The shuttle provided the adequate stiffness to insert the electrode to a target depth of at least 8 mm, without bending or buckling (FIG. 2C). Within a few minutes after implantation the PEG coating dissolves and the shuttle can be easily removed, leaving the electrode in place (FIGS. 2E-2F). This insertion procedure allows not only for accurate placement of the electrode, which is of a primary importance for the efficacy of stimulation therapies, but also for the minimization of acute damage to the brain tissue.

### Example 10

To characterize the acute reaction to the electrode, a group of rats (N=3) were implanted with electrodes bilaterally using a CNT fiber electrode, as described above, and a commercial PtIr stimulating microelectrode (75 to 25 μm diameter shaft with a blunt conical tip of approximately 25 μm and 5 μm maximum and minimum diameter, respectively; average impedance 10 kOhm; MicroProbes, Maryland, USA). They were then given an intravenous injection of a BBB-impermeable dye (DiI, Sigma Aldrich). Following the injection rats were immediately sacrificed and intracardially perfused with 4% paraformaldehyde, which served to fix the tissue as well as flush the dye from the vasculature. Thus, presence of the dye in the tissue is indicative of disruption of the BBB. Post-mortem acute histology shows that the bleeding around CNT fiber implant is comparable both as intensity and length scale with the PtIr electrode, even at the terminal site, where the size of PtIr is almost 10 times smaller than the complex CNT electrode-PI shuttle (FIGS. 2G-2H). It is hypothesized that the contained acute damage is due to the combined effects of CNT fiber flexibility and the presence of the PEG, which dissolves during the insertion and contributes to the reduction of the shear stress at the interface between the CNT fiber implant and the tissue.

### Example 11

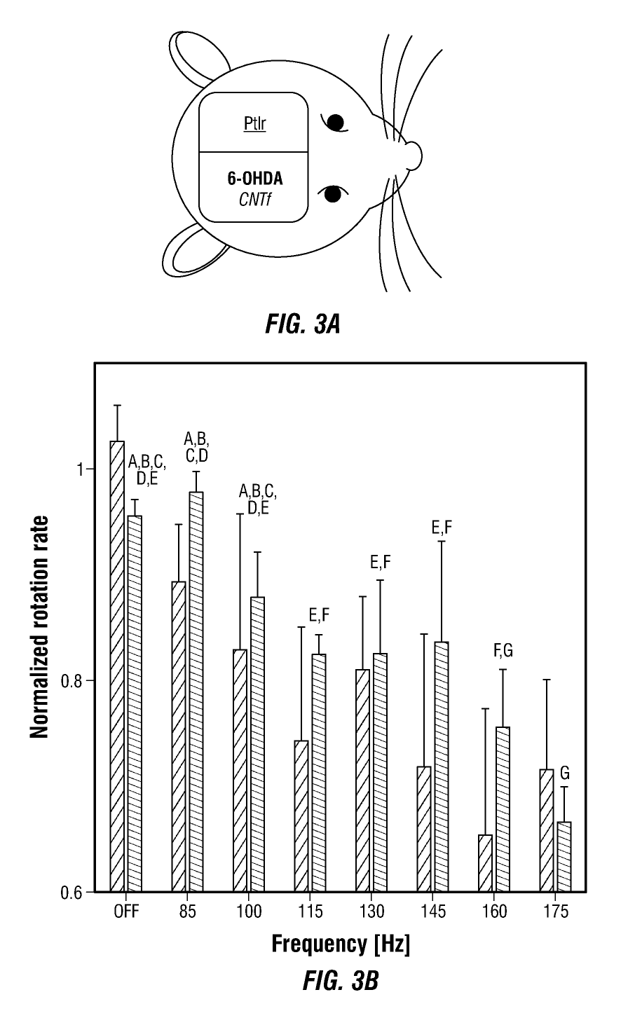

In vivo experimental studies in the rodent model of PD were then performed to evaluate the efficacy of CNTf stereotrodes as stimulating electrodes for DBS. This population of rats (N=4) was induced to be hemi-parkinsonian by receiving a unilateral injection of the neurotoxin 6-hydroxydopamine (6-OHDA) in medial forebrain bundle (MFB) for retrograde transport to substantia nigra pars compacta (SNc). The 6-OHDA selectively destroys the dopaminergic neurons SNc. The motor symptoms of PD result from the loss of dopaminergic neurons in the SNc and, thus, after the unilateral 6-OHDA lesion rats display similar gait and behavioral symptoms observed in patients with PD on the side of their body contralateral to the lesion. CNTf stimulating electrodes were implanted in the right entopeduncular nucleus (EP), the rat equivalent of the GPi. The same type of PtIr microelectrodes used for the acute studies were implanted contralaterally in the left EP and used as a control (FIG. 3A).

To assess the efficacy of the CNTf stereotrode for GPi-DBS, an amphetamine rotation test was performed, which is a commonly adopted behavioral test, used to quantify the effectiveness of the deep brain stimulation treatment in attenuating the motor asymmetry produced by the unilateral 6-OHDA lesion. Methamphetamine, a dopamine agonist, was administered intraperitoneally (I.P.) to the subjects (1.875 mg/kg; Sigma Aldrich) to induce locomotory rotations in the direction ipsilateral to the SNc lesion. The unidirectional rotation rate is an indicator of extent of the dopaminergic lesion, i.e. the extent of hemi-parkinson induced in the subject, as this circling behavior is not present if striatal dopamine is not depleted. Effective deep brain stimulation of the GPi allows for increased disinhibition of neural activity and the resultant behavior is closer to that in a healthy (unlesioned) state, which means that with therapeutic electrical stimulation the rotation rate will be attenuated. Thus, reduction in the rotation rate with deep brain stimulation indicates the efficacy of the therapy.

Electrical stimulation was administered at various frequencies during test epochs, which were interleaved with epochs without stimulation; these “off” periods were used to normalize the rotation rate during the test epoch since the baseline rotation changes over time as the methamphetamine is metabolized.

Deep brain stimulation with CNT fiber electrodes were able to significantly reduce the normalized metamphetamine-induced rotation rate (FIG. 3B). Moreover, it was also determined that the efficacy of treatment with CNT fiber electrodes improved as the frequency of the stimulating electrical current pulses was progressively increased from 85 to 130 Hz, thus replicating not only qualitatively but also quantitatively the modulation of motor-symptoms with deep brain stimulation previously observed using conventional PtIr. The CNT fiber microelectrode of the present disclosure is the smallest surface area electrode ever shown for successful alleviation of motor symptoms of PD via deep brain stimulation in any animal model.

### Example 12

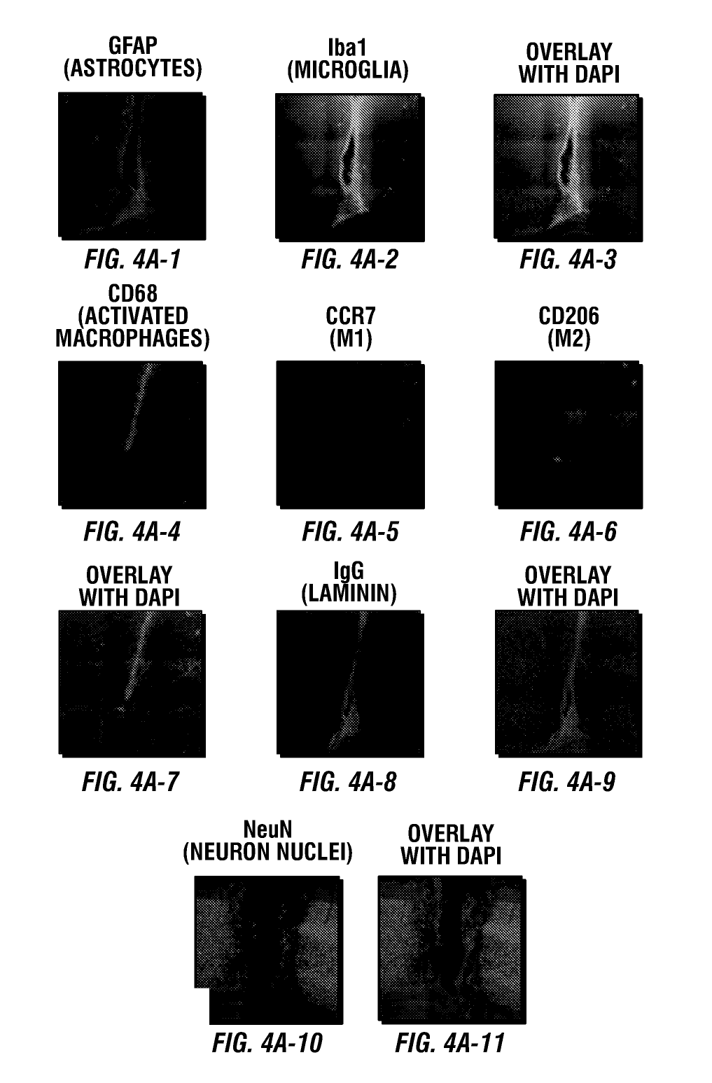

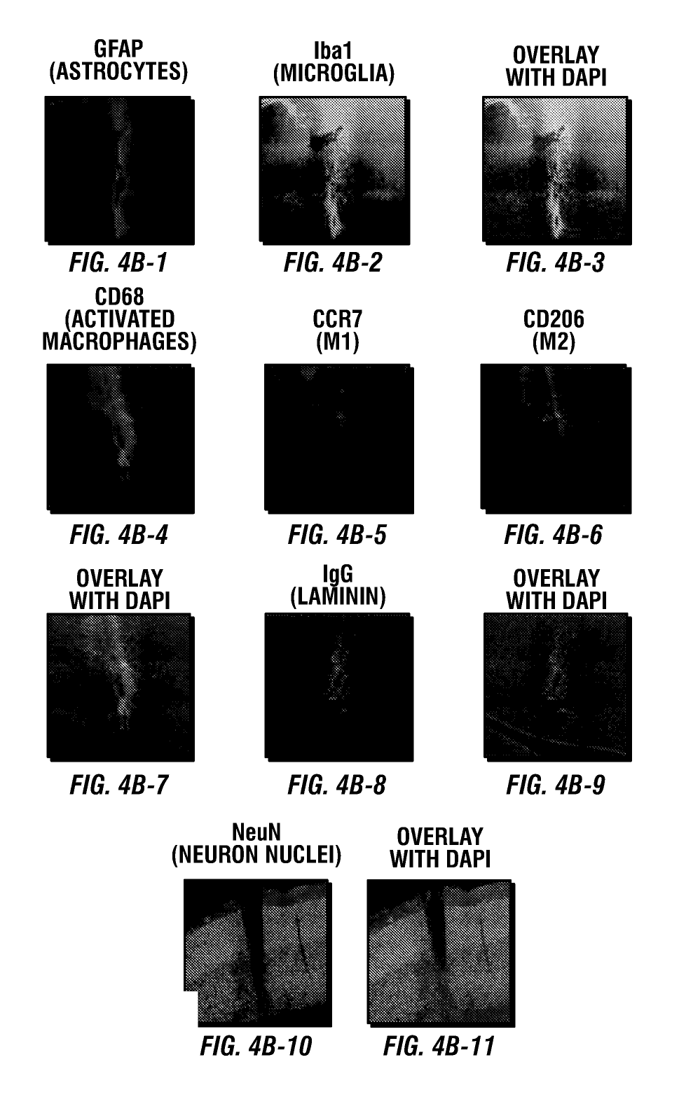

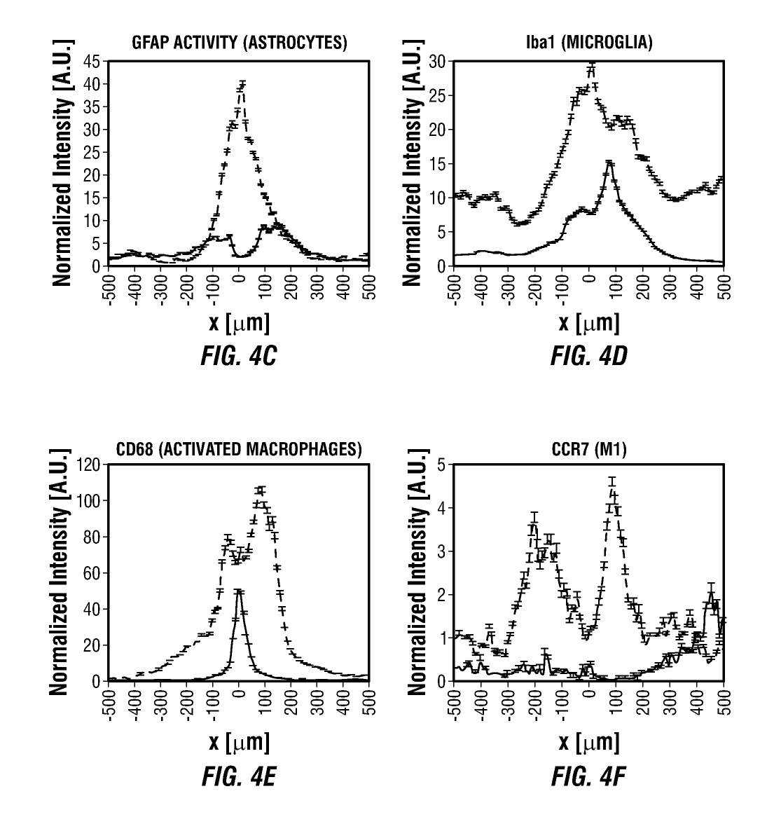

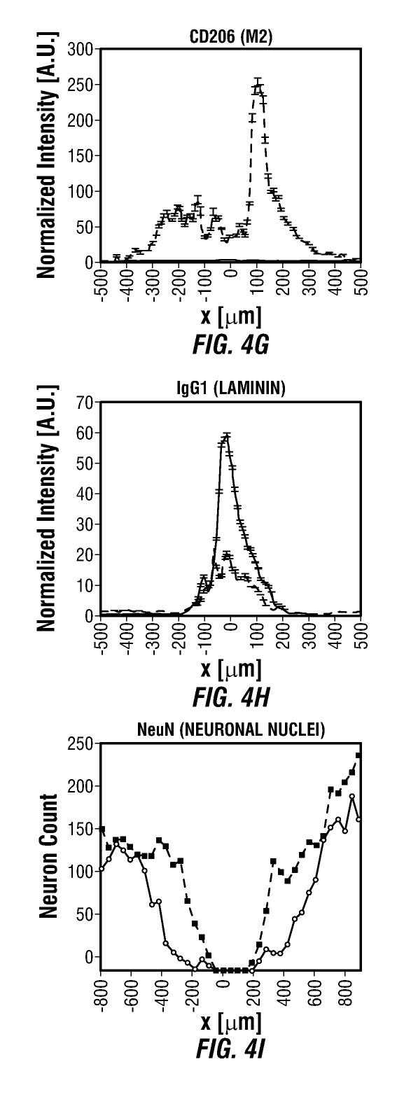

One of the major challenges in the design of neural interfaces is the minimization of the long-term, chronic reaction and improvement of the biocompatibility of the implant. The chronic inflammatory response is characterized by the neuronal cell loss and formation a dense encapsulating layer around the electrode, namely the glial scar, containing microglia/macrophages and astrocytes. The formation of this sheath causes the increase of the impedance of the tissue surrounding the electrodes, which in turn, causes the degradation of recording quality, loss of efficacy and possible dangerous voltage excursions at the stimulation site. Several studies show that the major factors affecting the extent of chronic inflammation are the electrode material, size and the relative micromotion between the electrode and the surrounding tissue. Stiff, bulky electrodes have been found to cause increase inflammatory response. The biocompatibility of CNTf electrodes use for deep brain stimulation was assessed six weeks post-implantation with immunohistochemistry analysis of central nervous system (CNS) inflammation and glial scar formation; results were compared with the PtIr microelectrodes implanted contralaterally (FIG. 4).

CNT fiber electrodes caused a four-fold reduction in the accumulation of astrocytes, as marked by the expression of glial fibrillar acidic protein (GFAP), and a two-fold reduction in the expression of general microglia, as marked by the expression of Iba 1, at the implant site, indicating a reduction in the reactive gliotic scar formation and electrode encapsulation (FIGS. 4A-4B, first row, and 4C-4D). Even more interesting results were observed for the analysis of the inflammatory response. Activated macrophages expression was found to be confined within approximately 50 μm adjacent to the implant and to be more than two times lower than at the site of the PtIr implant, where the zone of activation extended to more than 150 μm away from the implant. Recently, several studies have revealed the importance of the different macrophage phenotype in determining the effects of the inflammatory response. Depending on the nature and on the time-course of the injury, activated microglia/microphage can differentiate into predominantly ‘pro-inflammatory’ phenotype M1 or into ‘anti-inflammatory’ phenotype M2. M1 macrophages produce oxidative metabolites and proinflammatory cytokines that are toxic to the surrounding tissue and have neurodegenerative effects, whereas M2 phenotype has been found to promote angiogenesis, matrix remodeling and fibrosis. Thus, upregulated expression of M1 macrophages is an indication of active, neurotoxic inflammatory processes and upregulation of M2 expression can be indicative of tissue repair processes, but also of formation of fibrotic scar.

When stained for surface markers of M1 and M2 macrophages, a very low upregulation of both phenotypes could be observed at the site of CNT fiber implant; conversely, an evident increase with respect to background levels was observed around the PtIr electrodes, particularly in the case of the M2 phenotype (FIGS. 4A-4B, second row and 4E). These results could suggest a more extended fibrotic scar around the PtIr electrode, which is also consistent with the higher levels of GFAP and Ibal and the tissue encapsulation that was found when electrode was explanted after 6 weeks (FIG. 7A-7D). A weaker neuronal population was found in correspondence of CNT fiber implant, with a two time more extended zone of neurodegeneration in comparison with PtIr. We hypothesize that this was caused by the electrode implant procedure, where the footprint of the complex CNT fiber electrodes and PI stiffener was larger than the PtIr microelectrode.

The blood brain barrier (BBB) function is crucial for the regulation of tissue homeostasis and protection of neurons from exposure to neurotoxic blood serum proteins; moreover, damage to the BBB has been shown to correlate with degradation of electrode functions. The integrity of the BBB was observed by the amount of laminin, as this is normally excluded from healthy, uninjured brain tissue; the amount of laminin around the electrode was found to be higher in the case of CNT fiber electrode; however, the distribution of laminin is broader around the PtIr electrode with a characteristic length scale of fluorescence decrease of 100 μm, indicating a wider diffusion of the extravasation of blood serum proteins than caused by the CNT fiber electrode, where the length scale was found to be 60 μm (FIGS. 4A-4B, third row and 4H). Overall the results of the chronic histology analysis suggest that CNT fibers do not induce cytotoxic reactions. The flexibility and the reduced size, of the CNT fiber microelectrodes allow for an improvement of the overall biocompatibility of the device. The integrity of the explanted electrodes after 6 weeks of implant and deep brain stimulation experiment was assessed with SEM imaging: CNTf micro electrodes did not show any change in the structure, any sign of degradation at the stimulation site or crack in the insulation (FIG. 7A-7D)).

Neuronal activity was recorded in 2 Long Evans rats with 2 independently movable tetrodes targeting the region of the motor cortex M1. In each of the tetrode one channel was composed of CNTf, and the remaining three were made out of Nickel-Chromium wires (inner diameter: 13 μm), insulated with polyimide. One the days following the surgery, NSpike software was used to acquire neural activity data in the freely moving rats. The LFP signal was recorded on either one or all channels of the tetrodes at a sampling rate of 30 kHz. The signals were referenced to one tetrode that served as a designated reference electrode. This electrode was referenced to the ground screw, which is connected to the ground pin of the pre-amp. The reference electrode was selected based on a low baseline level of activity, which enabled a higher SNR signal to be acquired from the other electrodes. Additionally, threshold-crossing event waveforms from all channels were saved when activity on one channel exceeded a tetrode-specific threshold, which was set between 35 and 60 uA (depending on the quality of the signal). These waveforms are forty samples with a sampling rate of 10 kHz and were digitally filtered between 300 Hz and 6 kHz. Additional post-processing was done in Matlab, where individual units were identified from the threshold-crossing events by clustering spikes using peak amplitude and spike width.

From the foregoing description, one skilled in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure, which is defined in the following claims.

What is claimed is:

1. A device comprising:
at least one implantable microelectrode comprising at least one fiber of aligned carbon nanotubes partially coated with a layer of biocompatible insulating material, wherein at least one end of the fiber is uncoated.

2. The device of claim 1, wherein the at least one fiber of aligned carbon nanotubes is formed by wet-spinning or direct spinning.

3. The device of claim 1, wherein the aligned carbon nanotubes are single-walled carbon nanotubes.

4. The device of claim 1, wherein the biocompatible insulating material comprises polystyrene-polybutadiene.

5. The device of claim 1, wherein the uncoated end of the fiber is electrically active.

6. The device of claim 1, wherein the at least one implantable microelectrode has a specific interface impedance ranging from about 5 Mohm μm² to about 50 Mohm μm².

7. The device of claim 6, wherein the at least one implantable microelectrode has an average impedance of about 10² kOhm at 1 kHz.

8. The device of claim 1, wherein the at least one implantable microelectrode is a high capacitance electrode.

9. The device of claim 8, wherein the at least one implantable microelectrode has a charge storage capacity of about 310 mC/cm² to about 430 mC/cm².

10. The device of claim 1, wherein the at least one implantable microelectrode has a diameter ranging from about 8 μM to about 100 μM.

11. The device of claim 1, further comprising a removable inserting device attached to the implantable microelectrode.

12. The device of claim 11, wherein the removable inserting device is a polyimide wire.

13. The device of claim 11, wherein the removable inserting device is attached to the implantable microelectrode by a dissolvable coating.

14. The device of claim 13, wherein the dissolvable coating is a polyethylene glycol (PEG) coating.

15. The device of claim 13, wherein the removable inserting device is a polyimide wire.

16. The device of claim 15, wherein the polyimide wire is attached to the implantable microelectrode by a polyethylene glycol (PEG) coating.

17. The device of claim 11, wherein the at least one implantable microelectrode is a stimulating electrode.

18. The device of claim 11, wherein the at least one implantable microelectrode is a sensory electrode at a single neuron level.

19. A method of implanting an implantable microelectrode into a subject, said method comprising:
providing at least one implantable microelectrode, wherein the at least one implantable microelectrode comprises at least one fiber of aligned carbon nanotubes partially coated with a layer of biocompatible insulating material, wherein at least one end of the fiber is uncoated; and
implanting the at least one implantable microelectrode into the subject.

20. The method of claim 19, wherein the implantable microelectrode has specific interface impedance ranging from about 5 Mohm μm²to about 50 Mohm μm².

21. The method of claim 19, wherein the implantable microelectrode has an average specific interface impedance of about 10² kOhm at 1 kHz.

22. The method of claim 19, wherein the implantable microelectrode is a high capacitance electrode.

23. The method of claim 22, wherein the implantable microelectrode has a charge storage capacity of about 310 mC/cm² to about 430 mC/cm².

24. The method of claim 19, wherein the implantable microelectrode has a diameter ranging from about 8 μM to about 100 μM.

25. The method of claim 19, further comprising a step of attaching the implantable microelectrode to a removable inserting device.

26. The method of claim 25, wherein the removable inserting device is a polyimide wire.

27. The method of claim 25, wherein the removable inserting device is attached to the implantable microelectrode by a dissolvable coating.

28. The method of claim 27, wherein the dissolvable coating is a polyethylene glycol (PEG) coating.

29. The method of claim 25, wherein the implantable microelectrode is a stimulating electrode at a single neuron level.

30. The method of claim 28, wherein removal of the removable inserting device occurs by dissolution of the polyethylene glycol coating after implanting the at least one implantable microelectrode.

31. The method of claim 19, wherein the method is utilized to measure in vivo levels of brain chemicals.

32. The method of claim 19, wherein the implantable microelectrode is a sensory electrode at a single neuron level.

33. The method of claim 19, wherein the at least one implantable microelectrode is implanted into the peripheral nervous system of the subject.

34. The method of claim 19, wherein the at least one implantable microelectrode is implanted into the central nervous system of the subject.

35. The method of claim 25, wherein the at least one implantable microelectrode is implanted into the deep brain structures (DBS).

36. A method of fabricating an implantable microelectrode, said method comprising:
forming a fiber of aligned carbon nanotubes; and
partially coating the formed fiber of aligned carbon nanotubes with a layer of a biocompatible insulating material, wherein at least one end of the fiber remains uncoated.

37. The method of claim 36, wherein the step of forming the fiber of aligned carbon nanotubes comprises wet-spinning or direct spinning.

38. The method of claim 36, wherein the aligned carbon nanotubes are single-walled carbon nanotubes.

39. The method of claim 36, wherein the biocompatible insulating material comprises polystyrene-polybutadiene.

40. The method of claim 36, wherein the uncoated end of the fiber is electrically active.

41. The method of claim 36, wherein the implantable microelectrode has a specific interface impedance ranging from about 5 Mohm μm²to about 50 Mohm μm².

42. The method of claim 36, wherein the implantable microelectrode has an average specific interface impedance of about 10² kOhm at 1 kHz.

43. The method of claim 36, wherein the implantable microelectrode is a high capacitance electrode.

44. The method of claim 43, wherein the implantable microelectrode has a charge storage capacity of about 310 mC/cm² to about 430 mC/cm².

45. The method of claim 36, wherein the implantable microelectrode has a diameter ranging from about 8 μM to about 100 μM.

46. The method of claim 36, further comprising a step of attaching the implantable microelectrode to a removable inserting device.

47. The method of claim 46, wherein the removable inserting device is a polyimide wire.

48. The method of claim 46, wherein the removable inserting device is attached to the implantable microelectrode by a dissolvable coating.

49. The method of claim 48, wherein the dissolvable coating is a polyethylene glycol (PEG) coating.

50. The method of claim 36, wherein the implantable microelectrode is a stimulating electrode at a single neuron level.

51. The method of claim 36, wherein the implantable microelectrode is a sensory electrode at a single neuron level.
